(19) United States
(12) Patent Application Publication
(10) Pub. No.: US 2025/0352750 A1
(43) Pub. Date: Nov. 20, 2025

(54) APPARATUS FOR SOUND DAMPING FOR A VENTILATOR

(71) Applicant: Loewenstein Medical Technology S.A., Luxembourg (LU)

(72) Inventors: Angela GERLACH, Hamburg (DE); Kathleen VEERS, Kiel (DE); Alexander OSTER, Gettorf (DE)

(21) Appl. No.: 19/204,957

(22) Filed: May 12, 2025

(30) Foreign Application Priority Data
May 14, 2024 (DE) 10 2024 113 434.8

(51) Int. Cl. A61M 16/00; A61M 16/10

(52) U.S. Cl. CPC A61M 16/0066; A61M 16/1075; A61M 2205/42

(57) ABSTRACT

An apparatus for sound damping for a ventilator, and a ventilator comprising the apparatus. In addition to the apparatus, the ventilator comprises a respiratory gas inlet, a respiratory gas outlet and a respiratory gas path formed between the respiratory gas inlet and the respiratory gas outlet and comprising a blower configured to deliver a respiratory gas from the respiratory gas inlet to the respiratory gas outlet, such that the respiratory gas path has a suction side and a pressure side. The apparatus comprises: a chamber, which is configured to be arranged between the blower and the respiratory gas outlet, on the pressure side, in the respiratory gas path, and a tubular duct, which is designed to connect the chamber fluidically to the respiratory gas outlet.

### CROSS-REFERENCE TO RELATED APPLICATIONS

The present application claims priority under 35 U.S.C. § 119 of German Patent Application No. 10 2024 113 434.8, filed May 14, 2024, the entire disclosure of which is expressly incorporated by reference herein.

### BACKGROUND OF THE INVENTION

### 1. Field of the Invention

The invention relates to an apparatus for sound damping. This apparatus is designed for a ventilator. In addition, the invention relates to a ventilator comprising an apparatus for sound damping.

### 2. Discussion of Background Information

A ventilator is an apparatus for ventilating an individual with insufficient, disrupted or failed breathing capability and can be used both in domestic settings and in clinical settings. A ventilator can help with natural breathing and/or can take over ventilation. Ventilators can also act on the breathing of an individual in other ways, e.g. as part of respiratory therapy, oxygen supply or cough assistance. In addition, ventilators can also be used for diagnostic purposes.

A ventilator generally has at least one respiratory gas path with an inlet, an outlet and a respiratory gas drive (blower) for delivering a respiratory gas. To prevent the ventilator from being perceived as troublesome, the operating noises should be as low as possible. The operating noises are caused by sound waves, which are produced by the blower, by the air ducting and by cooling fans, for example. The prior art has disclosed ventilators which are equipped with sound-deadening foams to reduce the operating noises. These are formed from polyester-based polyurethane, for example. EP 3708208 A1, the entire disclosure of which is incorporated by reference herein, for example, shows an apparatus for ventilation with absorber foams for sound-deadening. However, it has been found that some foams may have a negative effect on the health of patients.

In view of the foregoing, it would be advantageous to have available an apparatus for a ventilator which offers effective sound suppression and ensures reliable and efficient operation of the ventilator.

### SUMMARY OF THE INVENTION

The present invention provides an apparatus for sound damping and by means of a ventilator as set forth in the independent claims. Further developments and advantageous refinements form the subject matter of the dependent claims, the features of which can be combined in any desired manner within the scope of what is technically feasible. This applies especially also beyond the limits of the various categories of claim. The description additionally characterizes and specifies the invention, in particular in conjunction with the drawings.

The invention relates to an apparatus for sound damping for a ventilator. In addition to the apparatus, the ventilator comprises a respiratory gas inlet, a respiratory gas outlet and a respiratory gas path, which is formed between the respiratory gas inlet and the respiratory gas outlet and has a blower, wherein the blower is designed to deliver a respiratory gas from the respiratory gas inlet to the respiratory gas outlet, such that the respiratory gas path has a suction side and a pressure side. The apparatus comprises a chamber, which is designed to be arranged between the blower and the respiratory gas outlet, on the pressure side, in the respiratory gas path, and a tubular duct, which is designed to connect the chamber fluidically to the respiratory gas outlet.

A “ventilator” can be understood to mean an apparatus for ventilation and/or for anesthetization. The ventilator can thus have a ventilation function and an anesthetic function, or a combination thereof. “Ventilation” can be understood to mean artificial ventilation, assistance with breathing, respiratory therapy, respiratory diagnosis, cough assistance, therapy involving the supply of oxygen, e.g. high-flow therapy, inhalation anesthesia, or a combination of at least two of these examples. Thus, an individual can be ventilated or assisted with breathing using the ventilator or, as an alternative or in addition, can also be kept under anesthetic. For this purpose, the ventilator can also be operated using anesthetic gases and can thus be used for inhalation anesthesia.

A ventilator can thus be understood to mean any device which assists an individual with natural breathing and/or take over ventilation and/or is used for respiratory therapy and/or is used for inhalation anesthesia and/or acts in some other way on the breathing of the patient or user. For example, a ventilator can be a ventilator for clinical or home applications, a respiratory therapy device, a CPAP, APAP or bilevel device, a high-flow therapy device, a narcosis or anesthesia device, an emergency ventilator, a device which supplies oxygen, a diagnostic system or a coughing therapy device or a cough assist machine.

The ventilator can have a housing. The respiratory gas inlet and the respiratory gas outlet of the ventilator can be arranged on the housing and/or can be formed by the housing. The respiratory gas path is formed between the respiratory gas inlet and the respiratory gas outlet, said path generally being situated—for the most part—within the housing. The respiratory gas path is set up and designed to carry a respiratory gas. “Respiratory gas” can be understood to mean a gas or gas mixture to be inhaled and/or exhaled. The respiratory gas can be normal breathable air from the surroundings, oxygen or breathable air enriched with oxygen. Respiratory gas can also be understood to mean a gas mixture which is enriched with at least one medicament or anesthetic, and therefore the respiratory gas can be used for therapeutic or anesthetic purposes.

The respiratory gas path can comprise at least one respiratory gas drive for guiding the respiratory gas in at least one direction. The respiratory gas drive can be embodied, for example, as a blower, in particular as a radial blower or as an axial blower, as a piston motor, as bellows or as a valve assembly. The respiratory gas drive is preferably embodied as a radial blower. The blower can have a suction nozzle and a pressure nozzle. In the case of radial blowers, the respiratory gas can enter the blower in the axial direction via the suction nozzle and leave the blower again via the pressure nozzle.

In some embodiments, the respiratory gas can leave the blower again perpendicularly to the axial direction via the pressure nozzle. In some embodiments, the respiratory gas may also be deflected in the pressure nozzle and/or downstream of the pressure nozzle. Thus, it is also possible in radial blowers for the respiratory gas to enter the blower in the axial direction, to be passed radially through the impeller and, after a deflection, to leave the blower again axially. For this purpose, the pressure nozzle can have a curvature, and/or the blower can have guide vanes which are configured to deflect the respiratory gas.

The blower generally comprises at least one rotatably mounted impeller, which is driven by a motor and produces a deflection of the respiratory gas from the axial to the radial direction. In this case, a flow of respiratory gas is produced. For ventilation, the blower can provide speeds of up to about 60,000 rpm or even above, thus enabling a corresponding respiratory gas pressure to be built up.

By means of the blower, the respiratory gas path can be divided into a suction side and a pressure side. The suction side is situated upstream of the suction nozzle of the blower. The respiratory gas can be fed into the respiratory gas path via the respiratory gas inlet and delivered to the blower on the suction side. The suction side is thus situated between the respiratory gas inlet and the suction nozzle of the blower. The pressure side is situated downstream of the pressure nozzle of the blower. Via the pressure side and the respiratory gas outlet, respiratory gas can be delivered from the blower to the patient. The pressure side is thus situated between the pressure nozzle of the blower and the respiratory gas outlet.

The ventilator can also have a plurality of inlets, via which, as an alternative or in addition, further respiratory gases, e.g. fresh gas, oxygen, gas mixtures, medicaments or anesthetics, can be introduced into the respiratory gas path. As an alternative or in addition, the ventilator can also have a plurality of outlets. The respiratory gas inlet and/or the respiratory gas outlet can form or comprise a connection for the respective connection of a hose or a hose system or a patient interface.

Via the respiratory gas outlet, the respiratory gas can be discharged from the respiratory gas path and supplied to the patient, e.g. via the hose or the hose system and/or via the patient interface. For the purposes of the invention, “patient interface” can be understood to mean any peripheral device which is designed for interaction with a living being. In particular, the patient interface is designed for respiratory, therapeutic and/or diagnostic purposes in combination with the ventilator. The patient interface can be designed as a breathing mask. This includes, without being exclusive, nasal masks, padded nasal masks, nasal cannulas or oxygen cannulas, full-face or total-face masks, as well as tracheal tubes and cannulas.

During the operation of the ventilator, operating noises may arise, these being produced, for example, by the blower, by the impeller, by the motor, by the air ducting or by cooling fans. This may be perceived as particularly troublesome by the patient if the operating noises occur or are present on the pressure side and may thus reach the patient directly. By means of the present invention, it is possible, in particular, to damp the pressure-side sound.

For the purposes of the invention, “sound damping” can be understood in general as reducing sound. Sound reduction can be accomplished by damping and/or insulation and/or acoustic impedances and/or reflections and/or sound-wave superposition (interference) and/or absorption. The term “sound damping” is used here as an overarching term for all physical effects which can reduce sound.

By means of the present invention, the sound is damped, in particular, by acoustic impedances. “Acoustic impedance”, in particular an acoustic flow impedance, designates the resistance which opposes sound propagation, for example in pipes. In the case of a transition between regions of different acoustic impedance, there is a reflection of the sound waves and, as a result, a sound-damping effect is achieved.

The apparatus according to the present invention having the chamber and the duct leads to a significant reduction in the operating noises coming from a ventilator. Since the chamber and the duct can be arranged on the pressure side of the respiratory gas path of a ventilator, it is possible, in particular, for the pressure-side sound arriving at the patient to be damped.

“Duct” can be understood, for example, to mean a pipe, a hose or a combination of both. The duct can be of rigid or at least partially flexible design. The duct is designed as an elongate cavity with a wall surrounding a lumen of the duct. Longitudinally, the duct has two openings, through which the respiratory gas can enter and leave the duct.

The duct is designed to receive and/or guide a respiratory gas. The duct has a length, a height and a width. In general, the length of the duct is many times greater than the width and/or the height. The height and the width of the duct can be the same or (slightly) different from one another. In the lumen of the duct, the respiratory gas can be received and/or guided. Driven by the respiratory gas drive of the ventilator, the respiratory gas can be guided through the duct. In this case, the through-flow direction generally corresponds to the longitudinal direction of the duct. Orthogonally with respect to the through-flow direction, the duct has a cross section.

“Chamber” can be understood to mean a region which has a different geometry from the duct. Like the duct, the chamber is designed to receive and/or guide a respiratory gas. The chamber is designed as a cavity with a wall surrounding a lumen of the chamber. The chamber can have two openings, through which respiratory gas can enter or leave the chamber. The chamber can have a chamber wall, the inner wall of which surrounds a lumen of the chamber. In the lumen of the chamber, the respiratory gas can be received and/or guided. At least in some parts, the chamber may also be formed by the ventilator itself, e.g. by parts of the housing, outer walls of other components of the ventilator and the like. The chamber has a length, a height and a width. In contrast to the duct, the length is generally not many times greater than the width and/or the height. Orthogonally with respect to the through-flow direction, the chamber has a cross section. The cross section of the chamber is generally larger than the cross section of the duct.

The respiratory gas path thus has different portions. The respiratory gas flows from the respiratory gas inlet to the blower on the suction side, and from the blower to the respiratory gas outlet on the pressure side. The blower can be understood as a portion of the respiratory gas path. Further portions, in particular ducts and chambers of different geometries and dimensions, can be arranged on the suction side and/or on the pressure side. One or more ducts and/or chambers are preferably formed on the pressure side. Arranging chambers and/or ducts on the suction side is likewise conceivable in some embodiments.

As a refinement, the apparatus may have a modular construction. Here, a module can correspond to one portion. Accordingly, one module can comprise a chamber, and another can comprise a duct etc. The modules can be designed in such a way that they can be arranged in series in different sequences. The modules can be designed as a plug-in system, for example, thus making it possible to achieve any order of the modules. This offers the advantage of a particularly flexible system. The modules can be arranged in such a way that, in the flow direction, the pressure nozzle is followed by a chamber, and the chamber is followed by a duct. This can be followed by additional ducts and chambers.

By virtue of the different portions (modules), the respiratory gas path undergoes at least one, preferably several, changes in cross section and/or changes in shape. The geometrical shape of the respiratory gas path is changed at least once, preferably several times. As a result, the cross section of the respiratory gas path can be reduced or increased at least once, preferably several times. It is preferred in this context if the cross section of the respiratory gas path is reduced or increased abruptly or stepwise at a transition between two portions (blower, chamber, duct). In one preferred embodiment, the transition between the portions is abrupt, giving rise to a clear geometrical discontinuity.

As a refinement, the cross section of the blower, in particular the cross section of the pressure nozzle, may be smaller than the cross section of the chamber. The cross section of the chamber may, in turn, be larger than the cross section of the duct. The cross sections of the pressure nozzle and of the duct may be the same or, preferably, different.

In order to achieve sound damping, at least one jump, preferably two, particularly preferably three or more, in the cross section is required. The more jumps in the cross section are present in the respiratory gas path, the greater is the sound-damping effect. At the same time, however, attention should be paid, as a refinement, to making the apparatus as small as possible since the different portions and/or cross sections entail a certain pressure loss. To compensate for this, the blower speeds can be adapted.

According to one embodiment, the blower may have a first acoustic impedance and the chamber can have a second acoustic impedance, and the duct may have a third acoustic impedance, wherein the acoustic impedances differ from one another in such a way that a sound produced by the blower is damped.

The apparatus is designed in such a way that there is preferably a step change in the impedances between the blower and the chamber. As an alternative or in addition, there can be a step change in the impedances between the chamber and the duct. As an alternative or in addition, there may also be a step change in the impedances between the blower and the duct. As described above, this is achieved by means of different geometries and/or cross sections.

According to one embodiment, the duct may comprise a duct wall with an inner wall surrounding a lumen of the duct. The inner wall of the duct may be designed to be constant. As an alternative or in addition, the chamber may comprise a chamber wall with a chamber inner wall surrounding a lumen of the chamber. The chamber inner wall may be designed to be constant.

“Constant” can be understood to mean that the duct wall and/or the chamber wall is continuously the same without any change or break. A constant wall is coherent, without any gaps, and continues uniformly—at least within one portion.

In some embodiments, the duct wall and/or the chamber wall may also be formed, at least in some parts, by the ventilator itself. Thus, for example, parts of the housing, outer walls of other components of the ventilator and the like can form the chamber wall or the duct wall, at least in part. However, it has proven advantageous for the chamber wall and the duct wall to be constant, ideally consisting of their own wall. At least in some portion or portions, the duct and the chamber may share the wall. This may mean that, in one operating state of the apparatus, i.e. in a state in which the modules adjoin one another, the duct wall simultaneously also forms the boundary of the chamber, and vice versa.

According to one embodiment, the apparatus may comprise a second chamber, which is arranged between the duct and the respiratory gas outlet. The second chamber can be designed to introduce another geometrical shape and/or another change in cross section into the respiratory gas path. The second chamber is preferably set up to form a stepwise increase or reduction in the cross section of the respiratory gas path. The second chamber can cause a further jump in the cross section of the respiratory gas path, thus reinforcing the sound-damping effect of the apparatus. Like the first chamber, the second chamber may comprise a chamber wall with a chamber inner wall surrounding a lumen of the chamber. The chamber inner wall of the second chamber may be designed to be constant.

According to one embodiment, the second chamber may have a fourth acoustic impedance, which differs from the first acoustic impedance and/or the second acoustic impedance and/or the third acoustic impedance, such that there is additional damping of a sound produced by the blower. The second chamber may be designed in such a way that there is a step change in the impedances between the second chamber and the duct and/or the chamber and/or the blower. Additional sound damping is thus achieved.

According to one embodiment, the duct and/or the chamber and/or the second chamber may be designed as a cavity in a nonporous material, in particular in a nonporous plastic. This means that the duct wall and/or the chamber wall comprise a nonporous material. Preferably, at least the duct inner wall and the chamber inner walls are produced from a nonporous material.

“Nonporous” can be understood to mean that the material is formed without (intentional) air inclusions, pores or the like. A nonporous material ideally has exclusively features of a solid state of aggregation. The material can be or comprise a metal and/or a nonporous polymer material (plastic), for example. The apparatus can be produced by means of casting, diecasting or the like, for example.

The material may comprise at least one of the following metallic materials: iron; steel, in particular stainless steel; aluminum; copper or other suitable metallic materials. Alternatively or in addition, the material may comprise at least one of the following polymeric materials: silicone, natural rubber, polyvinylchloride (PVC), polymethylmethacrylate (PMMA) or other suitable plastics.

The metallic materials may have a density of from about 2,000 to about 10,000 kg/m³, preferably of from about 2,700 to about 8,950 kg/m³. The metallic materials may have a hardness of from about 20 to about 120 HB. The metallic materials may have a tensile strength of from about 60 to about 950 N/mm².

The polymeric materials may have a density of from about 800 to about 1,500 kg/m³, preferably of from about 920 to about 1,400 kg/m³. The polymeric materials may have a Shore A hardness of from about 20 to about 100 Shore A. The polymeric materials may have a tensile strength of from about 1 to about 80 N/mm².

It has proven particularly advantageous that the respiratory gas path is formed substantially without foam and/or without nonwoven. This enables the respiratory gas path to be made particularly safe for the patient since no substances that are prejudicial to health can be formed in the respiratory gas path and/or released to the respiratory gas. Moreover, cleaning, disinfection or sterilization are made simpler and more reliable if the respiratory gas path is formed from a nonporous material since the nonporous material is not attacked by the cleaning agents to the same extent as foams.

As a preferred option, it is possible, in particular, for the duct and/or the chamber and/or the second chamber or, as a further preference, all the components of the ventilator along the respiratory gas path to be formed without foam and/or nonwoven.

Nevertheless, a filter, e.g. a pleated filter, or a filter system consisting of a plurality of filters arranged in series and/or a nonwoven may be arranged upstream of the respiratory gas path. The filter and/or the nonwoven may be arranged in or at the respiratory gas inlet of the ventilator. The filter and/or the nonwoven may be exchangeable. The filter and/or the nonwoven may be configured to filter respiratory gas flowing into the respiratory gas path via the respiratory gas inlet. The filter and/or the nonwoven may be configured to filter possible solid particles out of the respiratory gas.

According to one embodiment, the duct may have a cross section which is round or angular orthogonally to a longitudinal direction of the duct. The duct may have any desired cross section. According to the present disclosure, the cross section may be round or angular, e.g. circular, oval, quadrilateral, rectangular or polygonal. However, other cross sections are likewise possible. At least in some portion or portions, the cross section of the duct may also be slotted, crescent-shaped, cruciform, star-shaped, trapezoidal, X-shaped, L-shaped, U-shaped, V-shaped, T-shaped, elliptical or the like, for example. The cross section may be constant or may change along the longitudinal direction. In some embodiments, for example, the cross section may change its geometrical shape or increase and/or decrease in size. The duct preferably has a round or oval cross section since a round or oval cross section has the best flow properties.

In one preferred embodiment, the cross section remains constant along the longitudinal direction of the duct. In some embodiments, however, it is also conceivable for the duct to increase and/or decrease along its longitudinal direction.

According to one embodiment, the duct may be of at least partially straight and/or at least partially bent design when viewed in its longitudinal direction. In one simple embodiment, the duct is of straight design in its longitudinal direction. However, it may be advantageous if the duct is of at least partially bent design. Thus, the apparatus can be of particularly space-saving design. Furthermore, a bent duct can bring about an advantageous deflection of the respiratory gas, which may have a positive effect on sound suppression.

According to one embodiment, the duct may comprise at least one straight portion and at least one bent portion, wherein the bent portion has at least one bend, such that the duct is bent in its longitudinal direction. According to one embodiment, the bend can be of round or angular design. The bend may be at least about 10°, preferably at least about 45°. As a particular preference, the bend may be from about 45° to about 180°. According to one embodiment, the duct can be bent in a U shape and/or L shape at at least one location, preferably at at least two locations, in its longitudinal direction. A U-shaped bend enables the duct to be bent through 180° at at least one location. An L-shaped bend enables the duct to be bent through 90° at at least one location. The bend(s) can be of round or angular design.

According to one embodiment, the duct has a first straight portion, a second straight portion, and a bent portion connecting the first straight portion to the second straight portion, wherein the bent portion has a U-shaped bend, such that the longitudinal axes of the first straight portion and of the second straight portion are parallel to one another. This embodiment can be of particularly space-saving design.

According to one embodiment, the duct may comprise a first bent portion and a second bent portion, and a straight portion connecting the first bent portion to the second bent portion.

According to one embodiment, the duct may comprise a plurality of straight portions and bent portions, such that the duct has a meandering course in its longitudinal direction, at least in some portion or portions. “Meandering” can be understood to mean that the duct has a series of several bends. These can be arranged in a regular or irregular way, and therefore the duct can be of symmetrical or asymmetrical design. A meandering course offers the advantage of being particularly space-saving in combination with the greatest possible length of the duct. The bends can run in one plane and thus bend the duct in two dimensions. In some embodiments, it is also possible for the duct to be bent in another plane, and there are therefore one or more three-dimensional bends. The bends in the duct can extend in any conceivable direction. By virtue of the bends in the duct, the duct can be of symmetrical or asymmetrical design. The duct can have any desired spatial arrangement. However, it is advantageous if the duct does not exceed or fall below a certain volume and/or certain dimensions.

According to one embodiment, the duct may comprise a plurality of straight portions and bent portions and may be arranged in such a way that the duct at least partially forms the first chamber in that a wall of the duct forms a wall of the chamber, at least in some region or regions.

According to one embodiment, the duct may be arranged in a U shape around the chamber. Provision may also be made for the duct to comprise only bent portions, such that it is of oval, circular or spiral design. As an option, the duct formed in this way can be designed to wind around the chamber. This can represent a particularly space-saving embodiment.

According to one embodiment, the duct may have a volume of about 100 cm³-10,000 cm³, preferably of about 500 cm³-5,000 cm³. It has proven particularly advantageous for the duct to have a volume of about 800-4,500 cm³. In order to achieve this volume, the duct has a length and a diameter. According to one embodiment, the duct may have a length of about 1 mm-1,000 mm, preferably about 10 mm-500 mm. A length of about 10-350 mm has proven particularly advantageous. Moreover, the duct may have a diameter of about 1 mm-30 mm, preferably about 5 mm-20 mm, particularly preferably about 10-15 mm. The length of the duct entails significantly reduced sound emission on the pressure side.

According to one embodiment, the chamber and/or the second chamber may have a volume of about 2,000 cm³-40,000 cm³, preferably of about 4,000 cm³-20,000 cm³, particularly preferably of about 10,000-15,000 cm³. The sound-damping effect occurs particularly if the chamber or the second chamber is dimensioned as defined above. According to one embodiment, the chamber and/or the second chamber may have a depth of about 50 mm-120 mm, preferably of about 80 mm-100 mm and/or a height of about 10 mm-70 mm, preferably of about 20 mm-40 mm and/or a width of about 10 mm-100 mm, preferably of about 40 mm-60 mm.

The depth of the chambers should be understood to mean an extent of the chamber volumes in the flow direction. The width and the height of the chambers define a cross section of the chambers, which is oriented perpendicularly to the depth and thus to the flow direction of the respiratory gas.

The cross section of the chamber and of the second chamber may have any desired geometrical shape or cross section. According to the present disclosure, it may be angular, in particular rectangular. However, it does not have to be angular and, according to one embodiment of the present disclosure, it may also be round or oval. Both elongate and short configurations of the chamber are possible according to the present disclosure.

However, it has proven advantageous if the chamber has a certain depth, as defined above. If the selected depth were too small, the incoming respiratory gas flow would impinge directly on the opposite wall of the chamber, possibly leading to flow noises.

According to one embodiment, the ratio of the volume of the duct to the volume of the chamber and/or of the second chamber may be at least about 1:2, preferably at least about 1:4, particularly preferably about 1:10.

According to one embodiment, the chamber and the second chamber may be of identical design. According to one embodiment, the chamber and the second chamber may be of different dimensions, with the result that the acoustic impedances differ from one another. According to one embodiment, the second chamber and the duct may be of different dimensions, with the result that the acoustic impedances differ from one another.

“Dimensioning” can be understood to mean the geometrical shape and/or the size and/or the volume and/or the dimensions of the length/height/depth. Thus, for example, it is conceivable for the chamber and the second chamber to be of different sizes and thus of different volumes. In some embodiments, the chamber and the second chamber may also have the same volume but different dimensions of length and/or height and/or depth. It is also conceivable for the chamber and the second chamber to have different geometrical shapes, such that the chamber is of quadrilateral or polygonal design, for example, and the second chamber is round or oval, for example, and vice versa. The geometrical shapes can be of symmetrical or asymmetrical design. Thus, it is also conceivable, for example, for the chamber to be of symmetrical design and for the second chamber to be of asymmetrical design, and vice versa.

By means of different dimensioning of the two chambers, these may be detuned with respect to one another. This can mean that there is no wave configuration which fits precisely into the chamber and the second chamber. Thus, the chamber and/or the second chamber can be configured to ensure that the sound waves, the length of which fits precisely into the chamber geometry, cannot pass through the chambers. This enables the sound to be damped.

According to one embodiment, the apparatus may comprise a first connection and/or a second connection in order to connect the apparatus to the respiratory gas path of a ventilator. The respiratory gas path may be designed to guide a respiratory gas. The respiratory gas may follow the following respiratory gas path within the ventilator: the respiratory gas can enter the respiratory gas path via the respiratory gas inlet. From the respiratory gas inlet, the respiratory gas can enter the blower via the suction-side portion of the respiratory gas path and the suction nozzle. The blower delivers the conveying energy for the respiratory gas and conveys the respiratory gas via the pressure nozzle into the pressure-side portion of the respiratory gas path. There, the respiratory gas enters the apparatus via the first connection. First of all, the respiratory gas enters the chamber and flows through the latter. From the chamber, the respiratory gas enters the duct and flows through the latter in the longitudinal direction thereof. From the duct, the respiratory gas can leave the apparatus via the first connection. The respiratory gas can then be conveyed out of the ventilator via the respiratory gas outlet and conveyed to a patient (not shown).

The ventilator may comprise a humidifier, which is configured to humidify the respiratory gas with a liquid and/or to heat it. For this purpose, the humidifier may comprise a liquid reservoir. Furthermore, a heating device for heating the water, e.g. in the form of a heating rod, may be included. Moreover, a device for measuring the temperature of the liquid, e.g. in the form of an dip tube, may be included. The humidifier may thus be designed to provide heated water, over which respiratory gas can be guided and thus heated and/or humidified.

In some embodiments, the humidifier may include the apparatus for sound damping, at least in part. Thus, the chamber and/or the second chamber and/or the duct may be arranged in the humidifier or formed by the latter.

There are respective transitions between the blower and the chamber and/or between the chamber and the duct and/or between the duct and the second chamber. The transitions may be designed as an opening in the wall of at least one of the respective elements. Thus, the elements may simply adjoin one another, and the respiratory gas may flow from one element into the next. In some embodiments, it is also conceivable for one element to be designed to project into the next.

The chamber may directly adjoin the pressure nozzle of the blower, for example. Thus, for example, the pressure nozzle may directly adjoin an opening in the chamber wall. In some embodiments, the pressure nozzle may also be passed into the wall or through the wall of the chamber and may project into the chamber. There is preferably no additional element (chamber or duct or other cavity) between the blower and the chamber.

This situation can be similar with the transition between the chamber or the second chamber and the duct. The duct may directly adjoin the chamber or the second chamber. In some embodiments, the duct may also be passed into the wall or through the wall of the chamber or the second chamber and may project into the chamber.

The invention furthermore relates to a ventilator. The ventilator has a respiratory gas inlet, a respiratory gas outlet and a respiratory gas path, which is formed between the respiratory gas inlet and the respiratory gas outlet and has a blower, which is designed to deliver a respiratory gas from the respiratory gas inlet to the respiratory gas outlet, such that the respiratory gas path has a suction side and a pressure side. The suction side is formed between the respiratory gas inlet and a blower inlet, and the pressure side is formed between a blower outlet and the respiratory gas outlet. The ventilator comprises an above-describe apparatus for sound damping, wherein the apparatus is arranged in the respiratory gas path between the blower outlet and the respiratory gas outlet and is connected to the respiratory gas path via the first connection and the second connection.

### BRIEF DESCRIPTION OF THE DRAWINGS

Embodiments of the invention are described below with reference to the attached drawings. The invention is not restricted to the exemplary embodiments illustrated. Neither the description nor the FIG.s should be interpreted as limiting the scope of the invention.

In the drawings,

FIG. 1 shows a schematic illustration of a ventilator 1 comprising an apparatus 10 according to one embodiment of the invention.

FIG. 2 shows the apparatus 10 according to a first embodiment of the invention.

FIG. 3 shows illustrative embodiments of a duct 13 according to the invention.

FIG. 4 shows the apparatus 10 according to a second embodiment of the invention.

FIG. 5 shows the apparatus 10 according to a third embodiment of the invention.

FIG. 6 shows the apparatus 10 according to a fourth embodiment of the invention.

The drawings are purely schematic and in some cases not to scale. Where the same reference signs are used in different drawings, these reference signs denote features that are the same or have the same effect.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

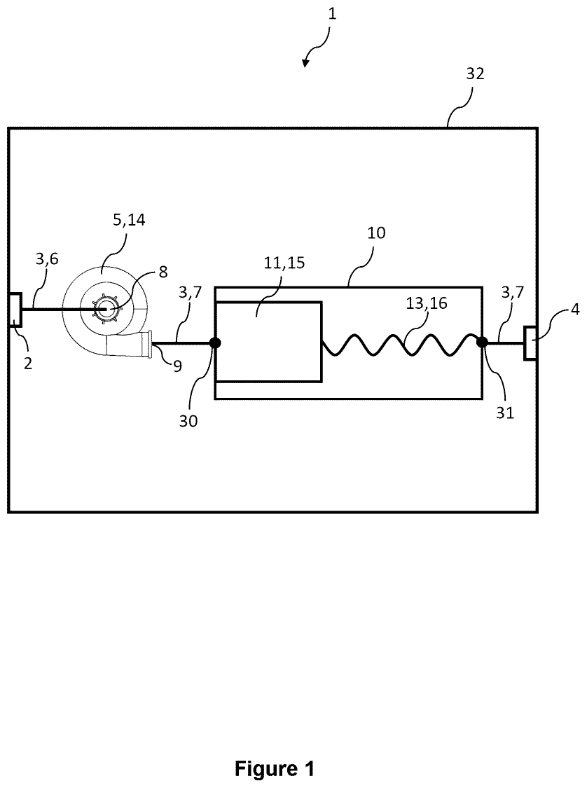

FIG. 1 shows a schematic illustration of a ventilator 1 comprising an apparatus 10 according to one embodiment of the invention. The ventilator 1 comprises a respiratory gas inlet 2, a respiratory gas outlet 4, and a respiratory gas path 3 formed between the respiratory gas inlet 2 and the respiratory gas outlet 4. The respiratory gas path 3 is designed to receive and/or guide and/or direct a respiratory gas.

As indicated schematically, the ventilator 1 can comprise a housing 32. The respiratory gas inlet 2 and the respiratory gas outlet 4 can be arranged on the housing 32 or be formed by the latter. It is also possible for the ventilator 1 to have a plurality of inlets 2 and/or outlets 4 (not shown). The respiratory gas inlet 2 and the respiratory gas outlet 4 can be situated opposite one another in the housing 32, as shown. However, the respiratory gas inlet 2 and the respiratory gas outlet 4 do not have to be situated opposite one another but can assume any suitable position on the ventilator. Via the respiratory gas inlet 2, a respiratory gas can be fed into the respiratory gas path 3. Via the respiratory gas outlet 4, a respiratory gas can be discharged from the respiratory gas path.

As illustrated schematically, at least one blower 5, which divides the respiratory gas path 3 into a suction side 6 and a pressure side 7, can be arranged in or on the respiratory gas path 3. The blower 5 can be designed as an axial blower or preferably, as shown, as a radial blower. The suction side 6 is formed between the respiratory gas inlet 2 and a blower inlet 8. The blower inlet 8 can be designed as a suction nozzle. The pressure side 7 is formed between a blower outlet 9 and the respiratory gas outlet 4. The blower outlet 9 can be designed as a pressure nozzle. By virtue of its geometry, the blower 5 can have a first acoustic impedance 14. The ventilator 1 furthermore comprises an apparatus 10 for sound damping. By virtue of its geometry, the apparatus 10 can have at least one further acoustic impedance 15, 16, 17, which differs from the first acoustic impedance 14.

The apparatus 10 is arranged on the pressure side 7 in the respiratory gas path 3. As an alternative or in addition, the apparatus 10 may also be arranged on the suction side 6 in the respiratory gas path 3. The apparatus 10 may comprise a first connection 30 and/or a second connection 31 in order to connect the apparatus 10 to the respiratory gas path 3 of a ventilator 1. The apparatus 10 then forms the respiratory gas path 3, at least in some region or regions, preferably the respiratory gas path 3 on the pressure side 7.

In this case, the first connection 30 can enable indirect or direct connection of the apparatus 10 to the blower 5. As shown, there can be a connecting line 3, 7 between the pressure nozzle 9 of the blower 5 and the first connection 30 of the apparatus 10, such that the apparatus 10 is connected indirectly to the blower 5. In some embodiments, the pressure nozzle 9 may also open directly into or at the first connection 30, such that the apparatus 10 directly adjoins the blower 5. The respiratory gas can then enter the apparatus 10 directly from the pressure nozzle 9 (not shown).

The second connection 31 can enable indirect or direct connection of the apparatus 10 to the respiratory gas outlet 4 of the ventilator 1. As shown, there can be a connecting line 3, 7 between the second connection 31 of the apparatus 10 and the respiratory gas outlet 4, such that the apparatus 10 is connected indirectly to the respiratory gas outlet 4. In some embodiments, the second connection 31 may also open directly into the respiratory gas outlet 4, such that the apparatus 10 directly adjoins the respiratory gas outlet 4. The respiratory gas can then leave the apparatus 10 directly from the ventilator 1 (not shown).

The apparatus 10 comprises a chamber 11, which is arranged between the blower outlet 9 and the respiratory gas outlet 4. The chamber 11 can have a second acoustic impedance 15. The geometric shape and the cross section of the chamber 11 is different from that of the blower 5. Thus, the first acoustic impedance 14 can differ from the second acoustic impedance 15 in such a way that a sound produced by the blower 5 is damped.

Furthermore, the apparatus 10 comprises a duct 13, which is arranged between the chamber 11 and the respiratory gas outlet 4. The duct 13 has a smaller diameter than the chamber 11. The duct 13 can have a third acoustic impedance 16. Since the geometric shape and the cross section of the duct 13 is different from that of the chamber 11 and/or of the blower 5, the third acoustic impedance 16 differs from the first acoustic impedance 14 and/or from the second acoustic impedance 15, such that there is additional damping of a sound produced by the blower 5.

The respiratory gas can follow the following respiratory gas path 3 within the ventilator 1: the respiratory gas can enter the respiratory gas path 3 via the respiratory gas inlet 2. From the respiratory gas inlet 2, the respiratory gas can enter the suction side 6 of the respiratory gas path 3 and, via the suction nozzle 8, the blower 5. The blower 5 delivers the conveying energy for the respiratory gas and conveys the respiratory gas via the pressure nozzle 9 into the pressure side 7 of the respiratory gas path 3. There, the respiratory gas enters the apparatus 10. First of all, the respiratory gas enters the chamber 11 and flows through the latter. From the chamber 11, the respiratory gas enters the duct 13 and flows through the latter in the longitudinal direction thereof. From the duct 13, the respiratory gas can leave the apparatus 10 and, via the respiratory gas outlet 4, the ventilator 1 and be conveyed to a patient (not shown).

At the transition from the blower 5 to the apparatus 10, more precisely from the blower 5 to the chamber 11, the geometry and/or the cross section of the respiratory gas path 3 changes, preferably stepwise. At the transition from the chamber 11 to the duct 13, the geometry and/or the cross section of the respiratory gas path 3 changes again, preferably stepwise. As a result, sound reflections occur, and therefore the sound produced by the ventilator 1 is damped. For this purpose, the ratio of the volume of the duct 13 to the volume of the chamber 11 can be at least 1:2, preferably at least 1:4, particularly preferably 1:10.

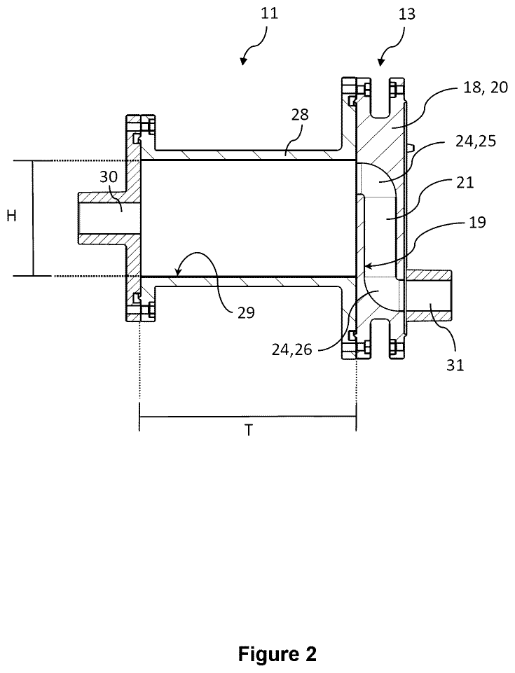

FIG. 2 shows the apparatus 10 according to a first embodiment of the invention. The apparatus 10 comprises the chamber 11 and the duct 13. FIG. 2 shows that the apparatus can have a modular construction. Here, a module can correspond to one portion 11, 12 of the apparatus 10. A first module can comprise the chamber 11, and another can comprise the duct 13. In this case, the modules can be designed as a plug-in system, and therefore the order of the modules can be of variable design. In some embodiments, it is also possible for a plurality of chambers 11 and ducts 13 to be arranged in series.

It is evident from FIG. 2 that the chamber 11 can comprise a chamber wall 28 with a chamber inner wall 29 surrounding a lumen of the chamber 11. The chamber inner wall 29 is preferably designed to be constant. The chamber inner wall 29 can be of uniform design, without breaks, corners, edges, gaps or the like. The chamber inner wall 29 is preferably of even and smooth design. It is thereby possible to enable particularly even, preferably laminar, flow without turbulence. Thus, there is a uniform flow pattern in the chamber 11, and this can have a positive effect on a pressure buildup in the respiratory gas.

Via the first connection 30, the respiratory gas can get into the lumen of the chamber 11 and flow through the latter. The chamber 11 has a depth T and a height H and a width that cannot be illustrated here. The depth T of the chamber 11 should not be too small since otherwise the incoming flow would strike directly as a free jet on the opposite wall. The opposite wall can preferably be a smooth wall without edges, or similar, in order to avoid flow noises.

It is furthermore evident from FIG. 2 that the duct 13 comprises a duct wall 18 with an inner wall 19 surrounding a lumen of the duct 13. The inner wall 19 is preferably designed to be constant. The duct 13 can be of at least partially straight design, when viewed in its longitudinal direction. The duct 13 may also be of at least partially bent design. The apparatus 10 can thus comprise a duct 13 which comprises at least one straight portion 21, 22 and at least one bent portion 25, 26. For this purpose, the bent portion 25, 26 has at least one bend 24, such that the duct 13 is of bent design in its longitudinal direction.

In the exemplary embodiment according to FIG. 2, the duct 13 has a first bent portion 25 and a second bent portion 26, and a straight portion 21 connecting the first bent portion 25 to the second bent portion 26. This embodiment has the advantage of being particularly space-saving. By virtue of the two bent portions 25, 26, the respiratory gas can furthermore undergo a deflection, thereby enabling additional suppression of sound. The bend 24 can be of rectangular design, for example, as shown. The bend 24 can thus be designed to deflect the respiratory gas in the bent portions 25, 26 through 90° in each case. Other angles for the bends 24 are conceivable.

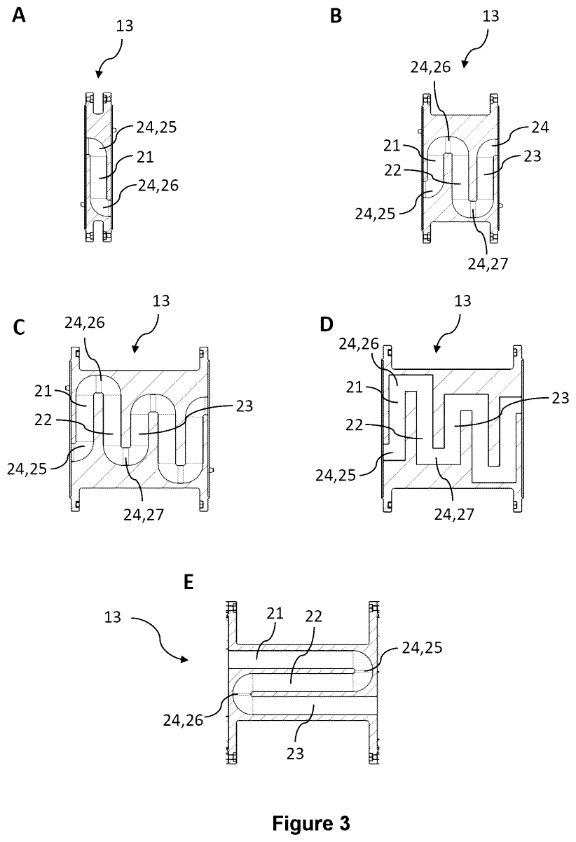

FIG. 3 shows illustrative embodiments of a duct 13 according to the invention. It is evident from FIG. 3 that the duct 13 can have many different shapes in respect of its longitudinal direction. In its simplest embodiment, the duct 13 can be of straight design (not shown).

The duct 13 can preferably have at least one bend 24, as shown in FIGS. 3A-E. The bend 24 can be of round design (FIGS. 3A-C, 3E) or angular (FIG. 3D).

For example, the duct 13 can have a U-shaped bend at at least one location, bending the duct through 180°. The duct 13 can also have an L-shaped bend at at least one location, bending the duct through 90°. The bend(s) can be of round or angular design.

By means of the bend 24, it is possible in principle to obtain any angle of the duct 13 in a 2- or 3-dimensional space. In preferred embodiments, however, the angle of the bend 24 can be at least 45°. The bend 24 can preferably bend the duct 13 between 45° and 180° in its longitudinal direction.

FIG. 3A shows an embodiment of the duct 13 having a first bent portion 25 and a second bent portion 26. The two bent portions 25, 26 are connected to one another by a straight portion 21. In this specific embodiment, the first bent portion 25 and the second bent portion each have an L-shaped bend 24, which amounts to 90°. In this case, the two bends 24 are of opposed design.

FIG. 3B shows an embodiment of the duct 13 with four bent portions 25, 26, 27 . . . , which can each be connected to one another by straight portions 21, 22, 23. By way of example, this embodiment has three straight portions 21, 22, 23. The straight portions 21, 22, 23 can be of the same length and/or have different lengths in the longitudinal direction thereof.

In this specific embodiment, the first bent portion 25 and the fourth, last, bent portion each have an L-shaped bend 24, which amounts to 90°. The second bent portion 26 and the third bent portion 27 each have a U-shaped bend 24, which amounts to 180°. By means of the bends 24 configured in this way, it is possible to arrange the longitudinal axes of each of the straight portions 21, 22, 23 parallel to one another. This has the advantage of being particularly space-saving.

FIGS. 3C and 3D show an embodiment of the duct 13 having a plurality of bent portions 25, 26, 27 . . . and a plurality of straight portions 21, 22, 23 . . . The duct 13 thus has a meanderingly convoluted course in its longitudinal direction. In this case, the longitudinal axes of the straight portions 21, 22, 23 can be arranged parallel to one another, as shown. Some other arrangement is conceivable.

The number of straight and bent portions selected can be variable. Here, it is the case that the more deflections the respiratory gas undergoes, the less noisy the apparatus 10 can be made. However, multiple deflections of the respiratory gas can also result in a pressure loss. In the configuration of the duct 13, the advantages and disadvantages of the deflections should thus be weighed against one another. The exemplary embodiments shown here have proven to be ideal embodiments that ensure optimum sound suppression combined with an adequate pressure buildup.

FIG. 3E shows an embodiment of the duct 13 with two bent portions 25, 26. The bent portions 25, 26 have a U-shaped bend 24. In addition, this embodiment has three straight portions 21, 22, 23. The bent portions 25, 26 are each connected to one another by the straight portions 21, 22, 23.

In alternative embodiments, the duct 13 can also be of fully bent design and can, for example, be wound in a spiral (not shown). In yet other embodiments, which are not shown, any desired arrangement consisting of 2- and 3-dimensional bends 24 can be provided, thus giving a symmetrical or asymmetrical arrangement of the duct 13.

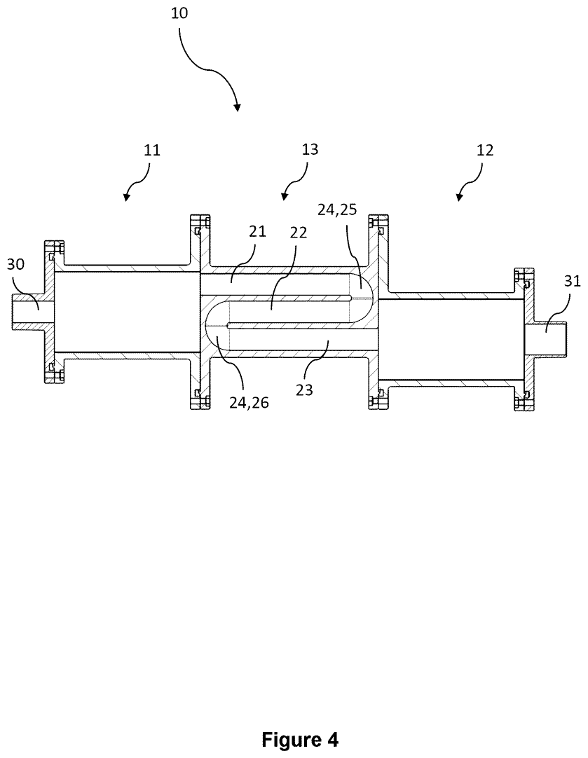

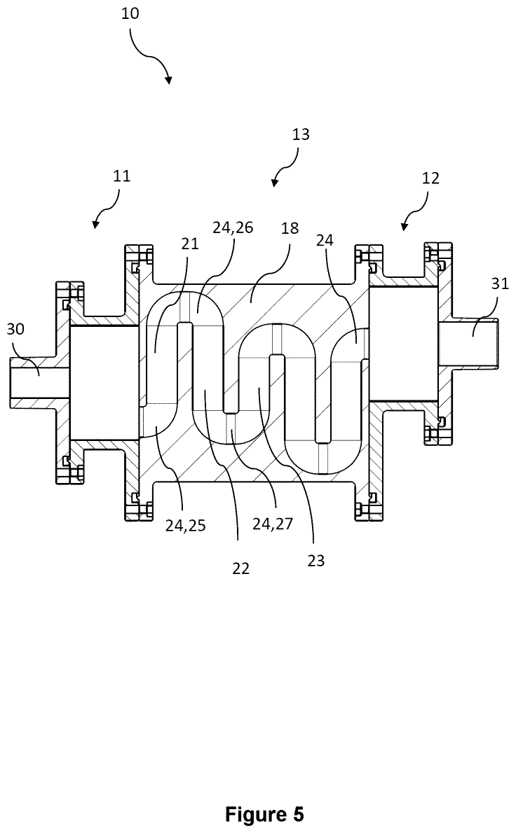

FIG. 4 shows the apparatus 10 according to a second embodiment of the invention, and FIG. 5 shows the apparatus 10 according to a third embodiment of the invention. These show that, in addition to the chamber 11 and the duct 13, the apparatus 10 can have a second chamber 12. The second chamber 12 is preferably arranged between the duct 13 and the respiratory gas outlet 4. The second chamber 12 can have a fourth acoustic impedance 17, which differs from the first acoustic impedance 14 and/or the second acoustic impedance 15 and/or the third acoustic impedance 16. As a result, a sound produced by the blower 5 can be additionally damped.

The chamber 11 and the second chamber 12 can be of identical dimensions and have identical geometrical shapes, as shown by way of example in FIGS. 4 and 5. In some embodiments, however, it may be advantageous for the chamber 11 and the second chamber 12 to have different dimensions and/or geometrical shapes (not shown). This makes it possible to ensure that the second acoustic impedance 15 (of the chamber) and the fourth acoustic impedance 17 (of the second chamber) differ from one another.

The chamber 11 and/or the second chamber 12 can be a humidifier or comprise a humidifier (not shown). The humidifier can be configured to humidify the respiratory gas with a liquid and/or to heat it.

FIG. 4 shows that the duct 13 can comprise a first straight portion 21, a second straight portion 22 and a third straight portion 23. Moreover, the duct 13 comprises a first bent portion 25, which connects the first straight portion 21 fluidically to the second straight portion 22. In addition, the duct 13 can comprise a second bent portion 26, which connects the second straight portion 22 fluidically to the third straight portion 23. In this case, as shown, the bent portions 25, 26 can have bends 24 such that the longitudinal axis of the first straight portion 21 is parallel to the longitudinal axis of the second straight portion 22, and/or the longitudinal axis of the second straight portion 22 is parallel to the longitudinal axis of the third straight portion 23.

The duct 13 can thus be bent at least once in a U shape in its longitudinal direction (not shown), preferably at least twice, as shown in FIG. 4. In other preferred embodiments, the duct can also be bent at least four times, or even more frequently (not shown), in a U shape.

FIG. 5 shows that the duct 13 comprises a plurality of straight portions 21, 22, 23 . . . and bent portions 25, 26, 27 . . . , such that the duct 13 has a meanderingly convoluted course in its longitudinal direction. As shown, the straight portions 21, 22, 23 . . . can have parallel longitudinal axes. The bends 24 of the bent portions 25, 26, 27 . . . can have the same and/or different angles. In the specific embodiment according to FIG. 5, the first bent portion 24 and the last bent portion each have an L-shaped bend 24 through 90°. The other bent portions 26, 27 . . . each have U-shaped bends, such that the longitudinal axes of the straight portions 21, 22, 23 are parallel to one another.

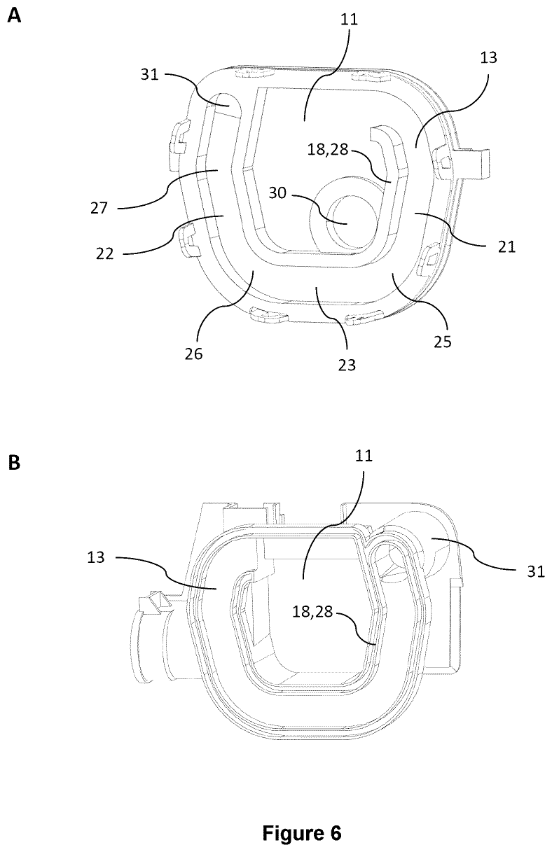

FIG. 6 shows the apparatus 10 according to a fourth embodiment of the invention, wherein the views in FIGS. 6A and 6B show the respective internal view according to a longitudinal section through the apparatus 10.

From this specific exemplary embodiment, it is evident that the duct 13 can comprise a plurality of straight portions 21, 22, 23 . . . and bent portions 25, 26, 27 . . . . It is furthermore evident from this Figure that the duct 13 and the chamber 11 can be formed by the same wall 18, 28, at least in some region or regions. The duct 13 can be arranged in such a way that it is formed at least partially around the first chamber 11 and at least partially surrounds the lumen of the chamber 11. The duct 13 can be designed to be bent in such a way that, overall, it is arranged in a U shape around the chamber 11. In alternative embodiments that are not shown here, provision can also be made for the duct to comprise only bent portions, such that it is of oval, circular or spiral design and, for example, is designed to wind around the chamber. This has the advantage of a particularly space-saving embodiment.

The respiratory gas can be directed into the chamber 11 via the connection 30. From this chamber, the respiratory gas can be directed into the duct 13, wherein the respiratory gas can be deflected through 90°. By virtue of the stepwise reduction or increase in the cross section of the respiratory gas path from the connection 30 to the chamber 11 and from the chamber 11 to the duct 13, effective sound damping can take place. The deflection of the respiratory gas can also have a sound-damping effect.

The respiratory gas can be guided directly from the duct 13 to the connection 31. It is also conceivable for a second chamber 12 to be arranged downstream of the duct 13 (not illustrated here). In some embodiments, this chamber can be arranged in a humidifier. FIGS. 2, 4, 5 and 6 each show embodiments in which there are respective transitions between the chamber and the duct and/or between the duct and the second chamber, said transitions being designed as a simple opening in the wall. The elements, namely the chamber, the duct and the second chamber adjoin one another, and the respiratory gas can flow from one element into the next. In alternative embodiments, it is also conceivable for one element to be designed to project into the next. The duct can then also be passed into the wall or through the wall of the chamber or the second chamber and can project into the chamber (not shown).

As shown, the apparatus can comprise the connections 30, 31 in order to connect the apparatus to the respiratory gas path 3 of a ventilator. Via the first connection, the apparatus can indirectly or directly adjoin the pressure nozzle of the blower at an upstream location. Via the second connection, the apparatus can indirectly or directly adjoin the respiratory gas outlet of the ventilator at a downstream location. As shown, the first connection and/or the second connection can be designed as nozzles, to which the respective components of the ventilator can be connected. In some embodiments, the connections may also be designed as a simple opening (not shown). Thus, for example, the pressure nozzle can directly adjoin an opening in the chamber wall or can be passed directly into the wall or through the wall of the chamber. In some embodiments, the pressure nozzle can project at least partially into the chamber. Moreover, the duct or, alternatively, the second chamber can have an opening which indirectly or directly adjoins the respiratory gas outlet of the ventilator, or forms said outlet.

Although the present invention has been described in detail with reference to the exemplary embodiments, it is self-evident for a person skilled in the art that the invention is not restricted to these exemplary embodiments. On the contrary, modifications are possible in such a way that individual features are omitted or different combinations of the individual features described can be implemented insofar as the scope of protection of the appended claims is not exceeded. The present disclosure includes all combinations of the individual features presented.

### LIST OF REFERENCE NUMERALS

1 ventilator
2 respiratory gas inlet
3 respiratory gas path
4 respiratory gas outlet
5 blower (respiratory gas drive)
6 suction side
7 pressure side
8 blower inlet (suction nozzle)
9 blower outlet (pressure nozzle)
10 apparatus for sound damping
11 chamber
12 second chamber
13 duct
14 first acoustic impedance
15 second acoustic impedance
16 third acoustic impedance
17 fourth acoustic impedance
18 duct wall
19 inner wall
20 material
21 first straight portion
22 second straight portion
23 third straight portion
24 bend
25 first bent portion
26 second bent portion
27 third bent portion
28 chamber wall
29 chamber inner wall
30 first connection
31 second connection
32 housing

What is claimed is:

1. An apparatus for sound damping for a ventilator, wherein, in addition to the apparatus, the ventilator comprises a respiratory gas inlet, a respiratory gas outlet and a respiratory gas path formed between the respiratory gas inlet and the respiratory gas outlet and comprising a blower which is configured to deliver a respiratory gas from the respiratory gas inlet to the respiratory gas outlet, such that the respiratory gas path has a suction side and a pressure side, the apparatus comprising:
a first chamber arranged between the blower and the respiratory gas outlet on the pressure side in the respiratory gas path,
a tubular duct configured to connect the chamber fluidically to the respiratory gas outlet.

2. The apparatus of claim 1, wherein the blower has a first acoustic impedance and wherein the first chamber has a second acoustic impedance, and wherein the tubular duct has a third acoustic impedance, the acoustic impedances differing from one another in such a way that a sound produced by the blower is damped.

3. The apparatus of claim 1, wherein the tubular duct is of at least partially straight and/or at least partially bent design when viewed in its longitudinal direction.

4. The apparatus of claim 1, wherein the tubular duct comprises at least one straight portion and at least one bent portion, the bent portion comprising at least one bend, such that the tubular duct is bent in its longitudinal direction.

5. The apparatus of claim 4, wherein the bend is of round or angular design and/or wherein the bend is at least 10°.

6. The apparatus of claim 1, wherein the tubular duct is bent in a U shape and/or L shape at at least one location in its longitudinal direction.

7. The apparatus of claim 1, wherein the tubular duct comprises a first straight portion, a second straight portion, and a bent portion connecting the first straight portion to the second straight portion, the bent portion having a U-shaped bend, such that longitudinal axes of the first straight portion and of the second straight portion are parallel to one another.

8. The apparatus of claim 1, wherein the tubular duct comprises a first bent portion, a second bent portion and a straight portion connecting the first bent portion to the second bent portion.

9. The apparatus of claim 1, wherein the tubular duct comprises a plurality of straight portions and bent portions, such that the tubular duct has a meandering course in its longitudinal direction, at least in some portion or portions.

10. The apparatus of claim 8, wherein the tubular duct comprises a plurality of straight portions and bent portions and is arranged in such a way that the tubular duct at least partially forms a first chamber in that a wall of the tubular duct forms a wall of the chamber, at least in some region or regions.

11. The apparatus of claim 10, wherein the tubular duct is arranged in a U shape around the chamber.

12. The apparatus of claim 1, wherein the apparatus further comprises a second chamber, which is arranged between the tubular duct and the respiratory gas outlet.

13. The apparatus of claim 12, wherein the second chamber has a fourth acoustic impedance, which differs from the first acoustic impedance of the blower and/or the second acoustic impedance of the first chamber and/or the third acoustic impedance of the tubular duct, such that there is additional damping of a sound produced by the blower.

14. The apparatus of claim 12, wherein the tubular duct and/or the first chamber and/or the second chamber configured as a cavity in a nonporous material.

15. The apparatus of claim 1, wherein the tubular duct has a volume of 100 cm³-10,000 cm³ and/or has a length of 1 mm-1,000 mm, and/or a diameter of 1 mm-30 mm.

16. The apparatus of claim 1, wherein the first chamber and/or a second chamber has a volume of 2,000 cm³-40,000 cm³.

17. The apparatus of claim 1, wherein the first chamber and/or a second chamber has a depth (T) of 50 mm-120 mm and/or a height (H) of 10 mm-70 mm and/or a width of 10 mm-100 mm.

18. The apparatus of claim 1, wherein the first chamber and/or a second chamber and/or the tubular duct are of different dimensions, such that the acoustic impedances differ from one another.

19. A ventilator, wherein the ventilator comprises a respiratory gas inlet, a respiratory gas outlet and a respiratory gas path formed between the respiratory gas inlet and the respiratory gas outlet and comprising a blower configured to deliver a respiratory gas from the respiratory gas inlet to the respiratory gas outlet, such that the respiratory gas path has a suction side and a pressure side, and wherein the ventilator further comprises the apparatus of claim 1, the apparatus being arranged in the respiratory gas path between the blower and the respiratory gas outlet and being connected in such a way, via a first connection and a second connection, to the respiratory gas path that the first chamber is arranged between the blower the respiratory gas outlet on the pressure side in the respiratory gas path, and that the tubular duct connects the first chamber fluidically to the respiratory gas outlet.

20. The ventilator of claim 19, wherein the ventilator further comprises a humidifier for humidifying and/or heating respiratory gas, the humidifier comprising the first chamber and/or the second chamber and/or the tubular duct.
